(19) United States
(12) Patent Application Publication
(10) Pub. No.: US 2026/0151529 A1
(43) Pub. Date: Jun. 4, 2026

(54) SCENT PRESENTATION MODULE, SCENT PRESENTATION SYSTEM, INFORMATION PROCESSING APPARATUS, AND SCENT PRESENTATION MODULE CONTROLLING SYSTEM

(71) Applicant: Sony Group Corporation, Tokyo (JP)

(72) Inventors: Shuji FUJITA, Tokyo (JP); Yukito INOUE, Tokyo (JP); Katsuhisa HAKODA, Tokyo (JP); Masahiro SHIMASE, Tokyo (JP)

(21) Appl. No.: 19/123,623

(22) PCT Filed: Jul. 16, 2023
(86) PCT No.: PCT/JP2023/026165

§ 371 Date: Apr. 23, 2025

(30) Foreign Application Priority Data
Dec. 2, 2022 (JP) 2022-193848

(51) Int. Cl. A61L 9/12

(52) U.S. Cl. CPC A61L 9/125; A61L 2209/11; A61L 2209/15

(57) ABSTRACT

To provide a technology that makes it possible to control scents flexibly.

A scent presentation module is provided that includes a scent holding section that holds a scent; an airflow forming section that forms a scent-containing airflow and that releases the airflow externally through a release opening of the scent presentation module; a controller that controls an operation of the airflow forming section; and a housing that includes therein the scent holding section, the airflow forming section, and the controller, the housing including a joining portion that enables the housing to be joined to another housing. Further, a scent presentation system that includes at least one scent presentation module is also provided, the scent presentation system presenting the scent using any of the at least one scent presentation module.

### TECHNICAL FIELD

The present technology relates to a scent presentation module, a scent presentation system, an information processing apparatus, and a scent presentation module controlling system. In particular, the present technology relates to a scent presentation module, a scent presentation system, an information processing apparatus, and a scent presentation module controlling system that make it possible to control scents flexibly.

### BACKGROUND ART

A technology that provides a scent to a user by supplying the air to a scent holding object that holds the scent and by releasing the scent using flow of the air, has been proposed in the past. For example, Patent Literature 1 discloses a scent apparatus that includes a scent head that can release gas containing a scent material and perform exhaustion for deodorization before the face of a user.

Further, in recent years, a scent is presented in connection with, for example, a video. For example, Patent Literature 2 discloses an olfactory display that presents a scent in a temporally and spatially limited area, the olfactory display including a housing that includes a nozzle; a plurality of scent rooms each including an air inlet and a scent outlet, the plurality of the scent rooms being formed by partitioning an internal space of the housing using partition walls; a scent passage that causes each of the scent outlets to communicate with the nozzle; a solid scent source that is accommodated in the scent room; a plurality of wind power sources each provided to a corresponding one of the plurality of scent rooms, each of the plurality of wind power sources forcing the air into the scent room through the air inlet using a diaphragm that includes a piezoelectric element; and an auxiliary wind power source that is provided to the housing separately from the plurality of wind power sources, the auxiliary wind power source accelerating, using the diaphragm including the piezoelectric element, the scent forced out of the scent room using the wind power source, the acceleration being performed in a direction in which the scent is injected.

### CITATION LIST

### Patent Literature

Patent Literature 1: Japanese Patent Application Laid-open No. 2005-304608
Patent Literature 2: Japanese Patent Application Laid-open No. 2014-230632

### DISCLOSURE OF INVENTION

### Technical Problem

However, there is more room for development for the needs of users with respect to, for example, fragrance apparatuses that are flexible in, for example, the types of handleable scents, the number of the scents, the number of the fragrance apparatuses that can be installed, and a location at which the fragrance apparatus can be installed. Actually, it is currently difficult to use the fragrance apparatuses freely for arrangement using scents.

Thus, it is a primary object of the present technology to provide a technology that makes it possible to control scents flexibly.

### Solution to Problem

First, the present technology provides a scent presentation module that includes a scent holding section that holds a scent; an airflow forming section that forms a scent-containing airflow and that releases the airflow externally through a release opening of the scent presentation module; a controller that controls an operation of the airflow forming section; and a housing that includes therein the scent holding section, the airflow forming section, and the controller, the housing including a joining portion that enables the housing to be joined to another housing.

Further, the present technology also provides a scent presentation system including at least one scent presentation module that includes a scent holding section that holds a scent; an airflow forming section that forms a scent-containing airflow and that releases the airflow externally through a release opening of the scent presentation module; a controller that controls an operation of the airflow forming section; and a housing that includes therein the scent holding section, the airflow forming section, and the controller, the housing including a joining portion that enables the housing to be joined to another housing, the scent presentation system presenting the scent using any of the at least one scent presentation module.

Furthermore, the present technology also provides an information processing apparatus that includes a terminal that is linked to a scent presentation module that includes a scent holding section that holds a scent, an airflow forming section that forms a scent-containing airflow and that releases the airflow externally through a release opening of the scent presentation module, a controller that controls an operation of the airflow forming section, and a housing that includes therein the scent holding section, the airflow forming section, and the controller, the housing including a joining portion that enables the housing to be joined to another housing; and a processor that receives information that is related to a state of the module and available to a user, that performs processing of outputting and storing the information, and that generates a signal related to the output and storing related to the information.

In addition, the present technology also provides a scent presentation module controlling system including at least one scent presentation module that includes a scent holding section that holds a scent, an airflow forming section that forms a scent-containing airflow and that releases the airflow externally through a release opening of the scent presentation module, a controller that controls an operation of the airflow forming section, and a housing that includes therein the scent holding section, the airflow forming section, and the controller, the housing including a joining portion that enables the housing to be joined to another housing; a terminal that is linked to the at least one scent presentation module; and an information processing apparatus including a processor that receives information that is related to a state of the module and available to a user, that performs processing of outputting and storing the information, and that generates a signal related to the output and storing related to the information.

### BRIEF DESCRIPTION OF DRAWINGS

FIG. 1 is a perspective view of a scent presentation module 1 according to a first embodiment of the present technology.

FIG. 2 is a set of six views of the scent presentation module 1 according to the first embodiment of the present technology.

FIG. 3 illustrates an internal structure of the scent presentation module 1 according to the first embodiment of the present technology.

FIG. 4 is a set of six views illustrating an embodiment of a scent holding section 10.

FIG. 5 is a perspective view of the embodiment of the scent holding section 10.

FIG. 6 is a cross-sectional view along the line P-P in the embodiment illustrated in FIG. 4.

FIG. 7 is a cross-sectional conceptual diagram illustrating how a scent is released from the scent holding section 10 illustrated in FIG. 4.

FIG. 8 is a perspective view of a scent presentation system 2 according to a second embodiment of the present technology when the scent presentation system 2 includes a single scent presentation module 1.

FIG. 9 is a perspective view of the scent presentation system 2 according to the second embodiment of the present technology when the scent presentation system 2 includes a single scent presentation module 1.

FIG. 10 is a perspective view of the scent presentation system 2 according to the second embodiment of the present technology when the scent presentation system 2 includes a single scent presentation module 1.

FIG. 11 is a perspective view of the scent presentation system 2 according to the second embodiment of the present technology when the scent presentation system 2 includes a single scent presentation module 1.

FIG. 12 is a perspective view of the scent presentation system 2 according to the second embodiment of the present technology when the scent presentation system 2 includes a plurality of scent presentation modules 1.

FIG. 13 is a perspective view of the scent presentation system 2 according to the second embodiment of the present technology when the scent presentation system 2 includes a plurality of scent presentation modules 1.

FIG. 14 is a perspective view of the scent presentation system 2 according to the second embodiment of the present technology when the scent presentation system 2 includes a plurality of scent presentation modules 1.

FIG. 15 is a perspective view of the scent presentation system 2 according to the second embodiment of the present technology when the scent presentation system 2 includes a plurality of scent presentation modules 1.

FIG. 16 is a flowchart illustrating processing performed by an information processing apparatus according to a third embodiment of the present technology.

FIG. 17 illustrates an embodiment of a UI displayed on a display section of a terminal.

FIG. 18 illustrates an example of a configuration of a scent presentation module controlling system 3 according to a fourth embodiment of the present technology.

FIG. 19 illustrates a modification of an internal layer portion 211.

FIG. 20 A of FIG. 20 is a cross-sectional view of the modification illustrated in FIG. 19, and B of FIG. 20 illustrates an example of a back surface of a lid portion 213 for internal layer portion.

FIG. 21 illustrates an example of an embodiment of a sealing container 50 used to seal in the modification illustrated in FIG. 19.

FIG. 22 illustrates a modification of a coupling portion 103.

FIG. 23 schematically illustrates an example of a silencing operation performed upon air discharge.

FIG. 24 schematically illustrates an example of a noise reducing operation performed upon air discharge.

FIG. 25 illustrates a modification of an airflow forming section 11.

### MODE(S) FOR CARRYING OUT THE INVENTION

Favorable embodiments for carrying out the present technology will now be described below with reference to the drawings.

Embodiments described below are examples of representative embodiments of the present technology, and the scope of the present technology is not construed as being limited to the embodiments. Note that the description is made in the following order.
1. First Embodiment (Scent Presentation Module 1)
(1) Example of Configuration of Scent Presentation Module 1
(2) Scent Holding Section 10
(2-1) Scent Carrier 101
(2-2) Venting Portion 102
(2-3) Coupling Portion 103
(2-4) Modification of Coupling Portion 103
(2-5) Others
(2-6) Example of Scent Release Operation Performed by Scent Holding Section 10
(3) Airflow Forming Section 11
(4) Modification of Airflow Forming Section 11
(5) Controller 12
(6) Housing 13
(7) Connection with External Unit
(8) Information Related to Scent Presentation Module 1
(9) Air Passage 15
(10) Others
2. Second Embodiment (Scent Presentation System 2)
3. Third Embodiment (Information Processing Apparatus)
4. Fourth Embodiment (Scent Presentation Module Controlling System 3)
5. Application of Scent Presentation Module 1 and Scent Presentation System 2

### 1. First Embodiment (Scent Presentation Module 1)

### (1) Example of Configuration of Scent Presentation Module 1

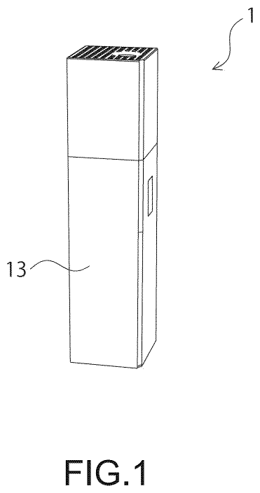

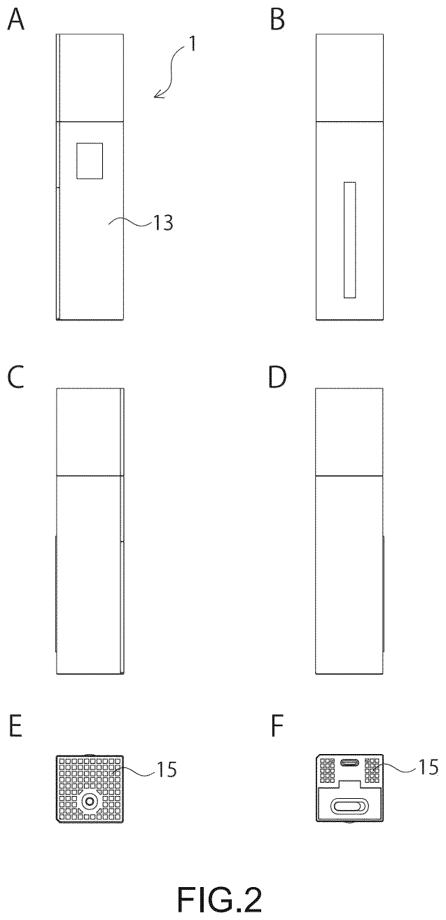

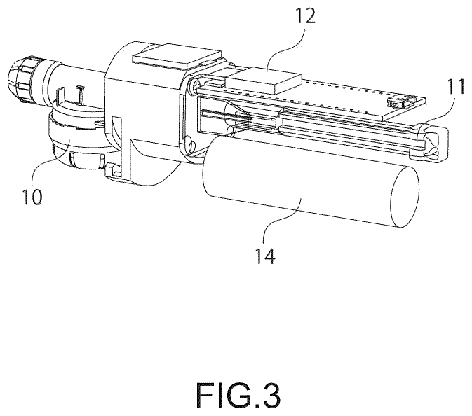

An example of a configuration of a scent presentation module 1 according to a first embodiment of the present technology is described with reference to FIGS. 1 and 2. FIG. 1 is a perspective view of the scent presentation module 1 according to the present embodiment. FIG. 2 is a set of six views of the scent presentation module 1 of the embodiment illustrated in FIG. 1, where A is a front view, B is a rear view, C is a left-side view, D is a right-side view, E is a plan view, and F is a bottom view. Further, FIG. 3 schematically illustrates an internal structure of the scent presentation module 1 according to the present embodiment.

The scent presentation module 1 according to the present embodiment includes a scent holding section 10, an airflow forming section 11, a controller 12, and a housing 13. Further, the scent presentation module 1 may include other structural elements such as a cell 14, a communication section (not illustrated), and an air passage 15, as necessary.

The scent presentation module 1 according to the present embodiment is described in detail below.

### (2) Scent Holding Section 10

The scent holding section 10 is a structural element that holds a scent. Note that, as used herein, the “scent” may include any components that stimulate a portion of or all of receptors such as odor molecules that exist in a nasal cavity. In addition to the olfactory receptors, there also exist, for example, trigeminal receptors in the nasal cavity, where the trigeminal receptor governs stimulations of, for example, coldness, hotness, and pain. The scent as used herein includes, in a broad concept, any components that stimulate a portion of or all of the receptors. Specifically, when, for example, menthol is used as a scent, the menthol also refers to stimulation provided through an olfactory receptor, and may also refer to coldness stimulation provided through a trigeminal receptor (a TRPA1 channel).

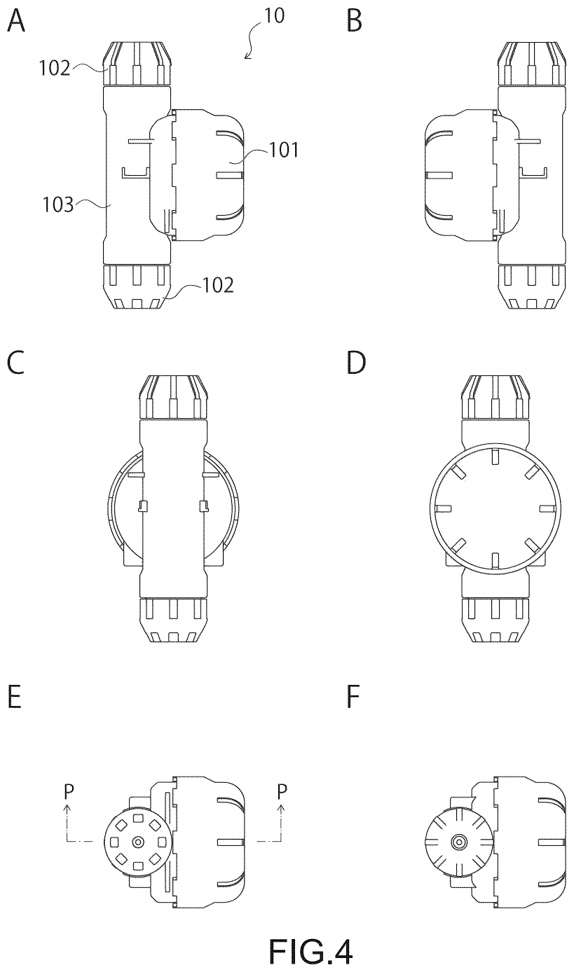

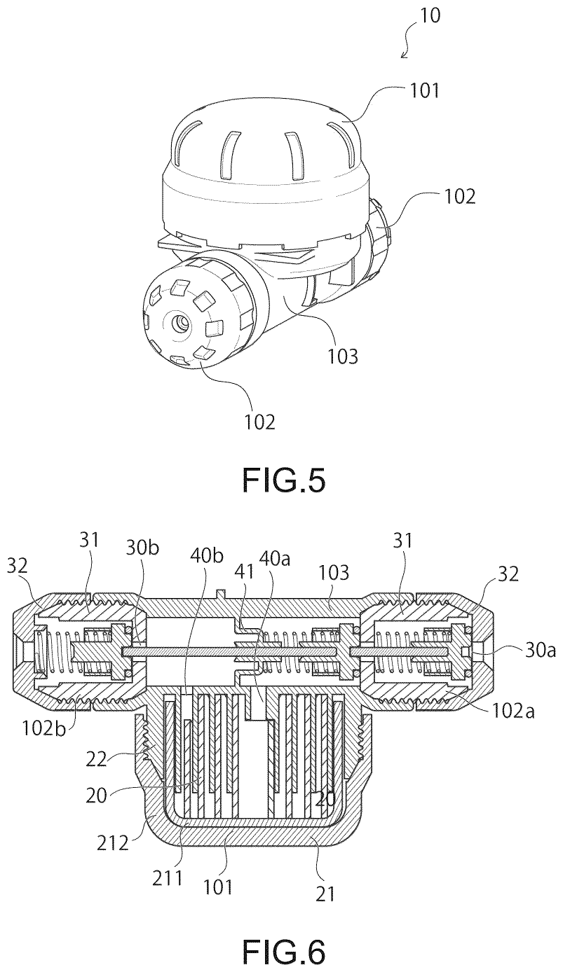

The scent holding section 10 may be, for example, a scent holding section of a replaceable cartridge type. FIG. 4 is a set of six views illustrating an embodiment of the scent holding section 10, where A is a front view, B is a rear view, C is a left-side view, D is a right-side view, E is a plan view, and F is a bottom view. FIG. 5 is a perspective view of the scent holding section 10 of the embodiment illustrated in FIG. 4, and FIG. 6 is a cross-sectional view along the line P-P in the embodiment illustrated in FIG. 4.

The scent holding section 10 according to the present embodiment includes a scent carrier 101, a venting portion 102, and a coupling portion 103. The adoption of this structure makes it possible to cause a scent to stay at a certain place, and thus to handle the scent easily. Further, the scent holding section 10 has a symmetric structure. The adoption of such a structure makes it possible to share a forming part, and thus to reduce costs.

Respective structural elements of the scent holding section 10 according to the present embodiment are described in detail below.

### (2-1) Scent Carrier 101

In the present embodiment, the scent carrier 101 includes an impregnation member 20 and a container portion 21. Further, in the present embodiment, a lid portion 22 corresponding to the container portion 21 is joined to a portion of the coupling portion 103 described later. Note that, in the present embodiment, the joining also includes a state in which the coupling portion 103 and the lid portion 22 are a single molded part.

Any material that can carry a scent may be adopted as a material used to form the impregnation member 20. Specifically, the impregnation member 20 may be made of an organic polymer material such that a scent can permeate the impregnation member 20 easily. For example, polyvinyl chloride, polyethylene, phenol resin, olefin resin, nylon, polyester, synthetic rubber, silicon resin, natural rubber, protein, nucleic acid, lipid, or polysaccharides may be used as the organic polymer material, or at least one thereof may be combined discretionarily to be used as the organic polymer material. Moreover, for example, polymer resin such as polycarbonate, acrylic resin, urethane resin, ABS resin, polyether ether ketone resin, polyacetal resin, fluororesin, cycloolefin polymer resin, or polyimide resin; metal such as stainless steel (steel use stainless (SUS)) or aluminum; an inorganic crystal such as quartz; glass; or filter paper may be used as the organic polymer material, or at least one thereof may be combined discretionarily to be used as the organic polymer material.

Further, the impregnation member 20 may be formed to be porous, and, for example, a mesh structure, a cork, mesoporous silica, or calcium carbonate may be used to form the impregnation member 20. Note that the impregnation member 20 may be formed to have a fiber structure or a layered structure (such as clay mineral).

The impregnation member 20 may also be in any form, and, for example, a sheet form, a net form, a strip form (also including densely collected strips), a particle form (also including densely collected particles), a gel-like form, a liquid form (also including liquid kept due to surface tension of, for example, a carrier body), a foam-like form, a three-dimensional structure (such as a pinholder form, a spiral form, or a spring form), or a string form (also including densely collected strings) may be used as the form of the impregnation member 20, or at least one thereof may be combined discretionarily to be used as the form of the impregnation member 20. When, for example, the impregnation member 20 is in a sheet form, the impregnation member 20 can be accommodated in the container portion 21 by being rolled into a cylindrical form, or by being folded in a multilayered formation. In the present technology, the impregnation member 20 may be in any form that enables the impregnation member 20 to be accommodated in the container portion 21.

Any component that can generate a scent may be adopted for a scent carried by the impregnation member 20. Specifically, for example, a liquid fragrance, a powder fragrance or the like with no change, a substance obtained by dissolving or dispersing the powder fragrance or the like into an appropriate solvent, refined oil, refined oil diluted with an appropriate solvent, fruit juice, food, or a substance obtained by dissolving or dispersing the food into an appropriate solvent may be used as the component of a scent, or at least one thereof may be combined discretionarily to be used as the component of a scent.

Favorably, the container portion 21 has a two-layer structure, and in the present embodiment, the container portion 21 includes an internal layer portion 211 that forms an inside portion of the container portion 21 that holds the impregnation member 20, and an external layer portion 212 that forms an outside portion of the container portion 21.

Any material that makes it possible to hold the impregnation member 20 may be used to form the internal layer portion 211. In the present embodiment, a portion of or all of the internal layer portion 211 is favorably formed using glass or metal. Examples of the metal include stainless steel (steel use stainless (SUS)) and aluminum. Further, in the present embodiment, an inner wall of the internal layer portion 211 that is situated on the side of the impregnation member 20 may be formed using metallic foil, or may be plated with metal. Any material may be used to form the external layer portion 212. For example, resin such as polyvinyl chloride, polyethylene, phenol resin, olefin resin, polycarbonate, acrylic resin, urethane resin, ABS resin, polyether ether ketone resin, polyacetal resin, fluororesin, cycloolefin polymer resin, or polyimide resin may be used to form the external layer portion 212, or at least one thereof may be combined discretionarily to be used to form the external layer portion 212.

Each of the internal layer portion 211 and the external layer portion 212 may also be in any form that enables a corresponding one of the portions to hold the impregnation member 20, and can be designed discretionarily. For example, a substantially cylindrical form, a substantially polygonal cylindrical form having a substantially polygonal cross section (a substantially triangular or rectangular cross section, or a cross section with five or more sides), a substantially conical form, or a substantially polygonal conical form having a substantially polygonal cross section (a substantially triangular or rectangular cross section, or a cross section with five or more sides) may be used as the form of each of the portions, or at least one thereof may be combined discretionarily to be used as the form of each of the portions.

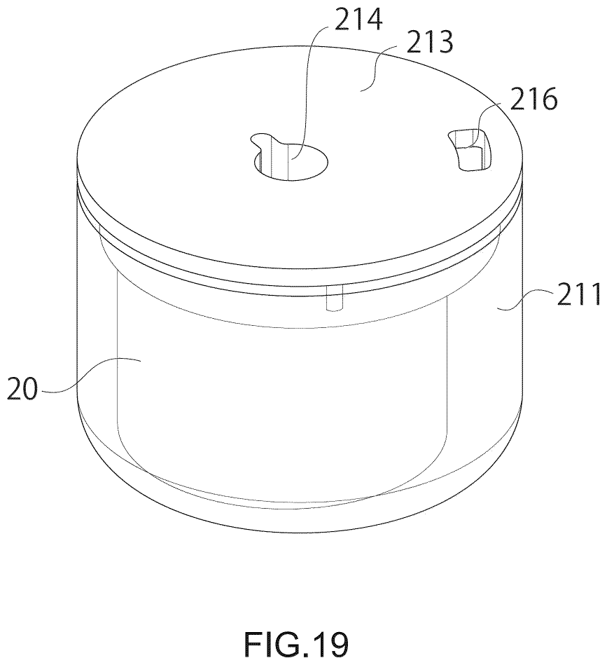

FIG. 19 illustrates a modification of the internal layer portion 211. In the modification illustrated in FIG. 19, the internal layer portion 211 further includes a lid portion 213 for internal layer portion that corresponds to the internal layer portion 211. Note that FIG. 19 does not illustrate the external layer portion 212. In the modification illustrated in FIG. 19, a structural element that carries a scent is independent of other structural elements. This makes it possible to only independently use the structural element carrying a scent. This provides an advantage in performing handling easily.

Favorably, a portion of or all of (favorably all of) the internal layer portion 211 is formed using a material exhibiting a high drug resistance and/or a less gas-permeable material. Further, a portion of or all of (favorably all of) the lid portion 213 for internal layer portion is favorably formed using a material exhibiting a high drug resistance. The formation using a material exhibiting a high drug resistance and/or a less gas-permeable material makes it possible to prevent a deterioration in a component that can generate a scent, and thus to achieve a long-term storage. Examples of the material exhibiting a high drug resistance and/or the less gas-permeable material include metal such as stainless steel (steel use stainless (SUS)) and aluminum, resin such as polytetrafluoroethylene (PTFE) and ethylene tetrafluoroethylene (ETFE), and glass. In the present embodiment, aluminum from among these materials is favorably used in particular.

In the modification illustrated in FIG. 19, the internal layer portion 211 and the lid portion 213 for internal layer portion can fit each other using a conventionally known fitting mechanism. Specific examples of the fitting include press fitting the lid portion 213 for internal layer portion into the internal layer portion 211; threading the internal layer portion 211 and the lid portion 213 for internal layer portion; using a cam latch handle or a lock part having, for example, a buckle structure; bonding using, for example, an adhesive; and thermal fusion bonding.

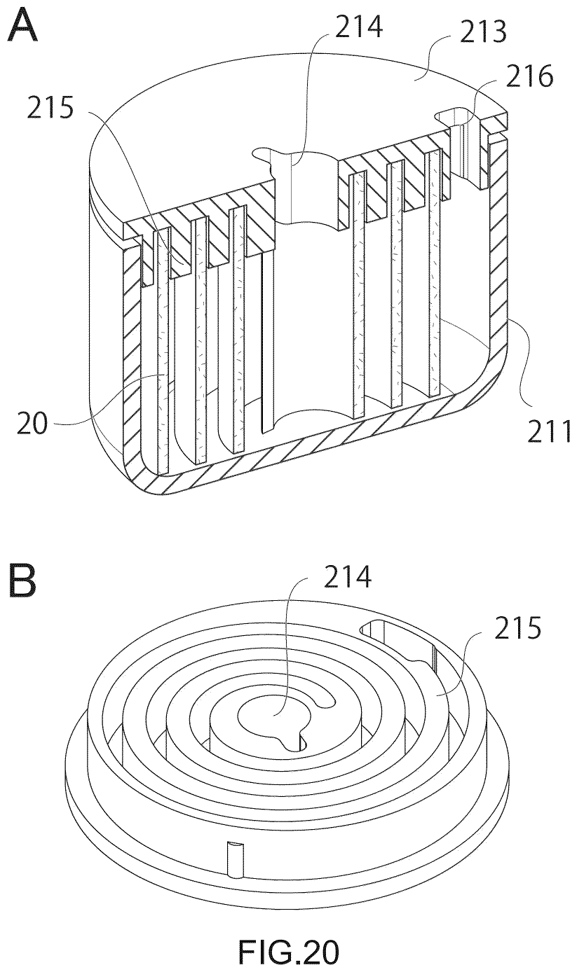

A of FIG. 20 is a cross-sectional view of the modification illustrated in FIG. 19, and B of FIG. 20 illustrates an example of a back surface of the lid portion 213 for internal layer portion. An airflow inlet 216 and an airflow outlet 214 are formed in the lid portion 213 for internal layer portion, as illustrated in FIG. 19 and A of FIG. 20. Further, in the present embodiment, the impregnation member 20 carrying a scent is in a sheet form, and is accommodated in the internal layer portion 211 by being rolled into a cylindrical form or by being folded in a multilayered formation. In this case, a spiral groove 215 is formed in the back surface of the lid portion 213 for internal layer portion, as illustrated in B of FIG. 20, such that the form of the impregnation member 20 remains unchanged. Further, the impregnation member 20 is favorably in contact with an upper surface of the lid portion 213 for internal layer portion and a bottom surface of the internal layer portion 211. As a result of adopting such a structure, airflow that flows into the internal layer portion 211 is discharged after passing through all of the spiral structure in the internal layer portion 211. This makes it possible to generate a scent-containing airflow with more certainty.

Further, the impregnation member 20 is set for the spiral groove 215 in advance as necessary, and this makes it possible to pour, through the inlet 214, a component that can generate, for example, a liquid scent or a semiliquid scent.

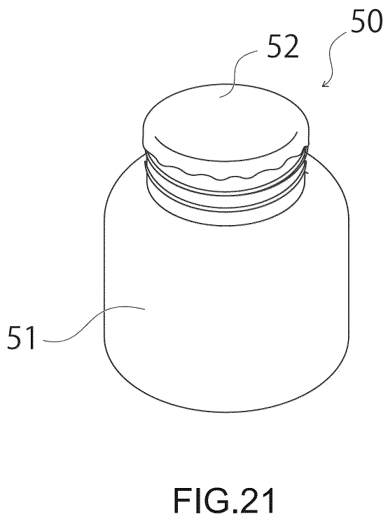

FIG. 21 illustrates an example of an embodiment of a sealing container 50 used to seal in the modification illustrated in FIG. 19. When, for example, the modification illustrated in FIG. 19 is sealed to be stored in the sealing container 50 illustrated in FIG. 21 until beginning of use (favorably, until just prior to usage), this makes it possible to prevent a deterioration in a component that can generate a scent. Further, a portion of or all of (favorably all of) a container portion 51 and a lid portion 52 for sealing container that are included in the sealing container 50 illustrated in FIG. 21 is favorably formed using a less gas-permeable material. Note that the less gas-permeable material has been already described above, and thus a description thereof is omitted here.

### (2-2) Venting Portion 102

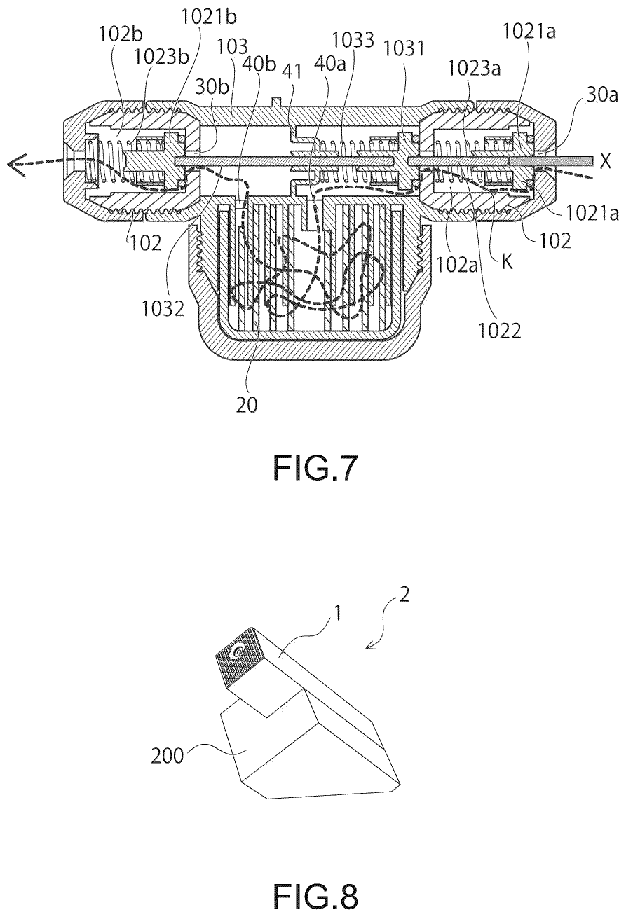

The venting portion 102 includes a vent 30 that can be opened and closed. In the present embodiment, the venting portion 102 is divided into two portions (a first venting portion 102a and a second venting portion 102b) with two vents 30, as illustrated in FIG. 7. The present technology is not limited thereto, and the venting portion 102 may be a single portion, or may be divided into at least three portions.

In the present embodiment, the venting portion 102 has a symmetric structure in which the venting portion 102 is divided into the two portions (the first venting portion 102a and the second venting portion 102b). The adoption of such a symmetric structure makes it possible to share a part for forming the two portions, and thus to reduce costs.

In the present embodiment, the vent 30 includes an inflow vent 30a and a release vent 30b, where the air flows into the scent holding section 10 through the inflow vent 30a, and a scent-containing airflow is released externally through the release vent 30b. An open-and-close mechanism communicates with each of the two vents 30 (30a and 30b). With respect to a specific structure of the open-and-close mechanism, any mechanism that can open and close each of the two vents 30 (30a and 30b) may be adopted, and the mechanism can be designed discretionarily. Specifically, an open-and-close mechanism that includes a sealing lid 1021 (1021a, 1021b), a shaft 1022, and a spring 1023 (1023a, 1023b) may be provided, as illustrated in FIG. 7.

In the present embodiment, the venting portion 102 favorably has a two-layer structure. In the present embodiment, the venting portion 102 includes an internal layer member 31 that stores therein the open-and-close mechanism, and an external layer member 32 that forms an outer portion of the venting portion 102. Any materials may be used to form the internal layer member 31 and the external layer member 32. Examples of the material include a material similar to the material used to form the above-described external layer portion 212, and thus, a description thereof is omitted here. Further, forms of the internal layer member 31 and of the external layer member 32 can be designed discretionarily according to, for example, the vent 30 or the open-and-close mechanism described above.

### (2-3) Coupling Portion 103

The coupling portion 103 includes at least two coupling openings 40 (40a and 40b) and a partition portion 41. The at least two coupling openings 40 are formed in the lid portion 22 joined to the coupling portion 103, and the at least two coupling openings 40 cause the scent carrier 101 to communicate with the lid portion 22. The partition portion 41 is arranged upstream from one of the at least two coupling openings 40 (40a and 40b).

In the present embodiment, the two coupling openings 40 (40a and 40b) include the first coupling opening 40a through which the air is released from the coupling portion 103 to the scent carrier 101, and the second coupling opening 40b through which a scent-containing air from the scent carrier 101 is released to the coupling portion 103. In this case, the air flowing into the coupling portion 103 through the first venting portion 102a is released to the scent carrier 101 through the first coupling opening 40a and mixed with a scent. Thereafter, a scent-containing air flows into the coupling portion 103 through the second coupling opening 40b and is released externally through the second venting portion 102b.

In the present embodiment, the partition portion 41 is arranged upstream from the second coupling opening 40b from between the two coupling openings 40 (40a and 40b). When the partition portion 41 is included, the air flowing into the coupling portion 103 is forced to pass through the scent carrier 101 to become a scent-containing air, and the air in the form of a scent-containing air flows into the coupling portion 103 again. This makes it possible to generate a scent-containing airflow efficiently.

Further, in the present embodiment, the coupling portion 103 includes an open-and-close mechanism in a connection region in which the coupling portion 103 is connected to the venting portion 102. The open-and-close mechanism is similar to the open-and-close mechanism described in “(2-2) Venting Portion 102”, and thus, a description thereof is omitted here.

### (2-4) Modification of Coupling Portion 103

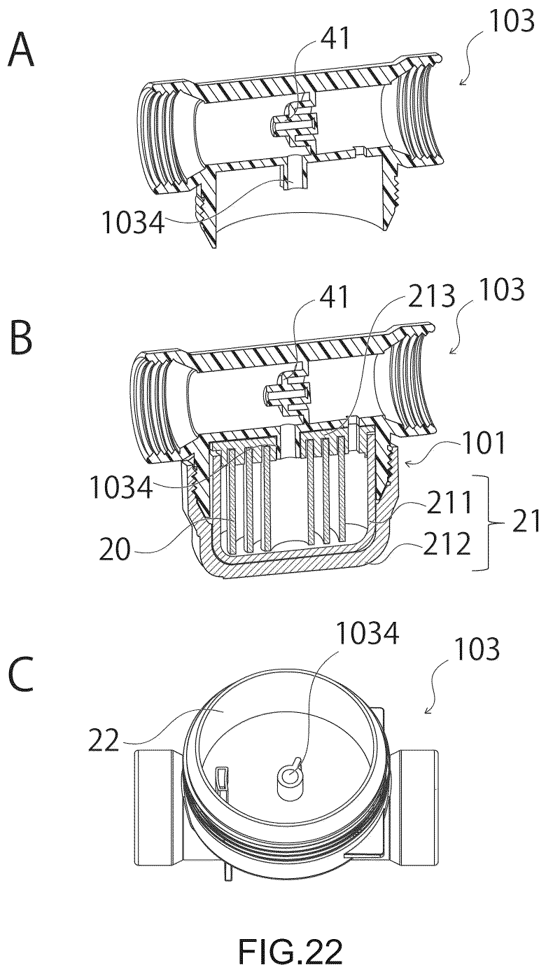

FIG. 22 illustrates a modification of the coupling portion 103. A of FIG. 22 is a cross-sectional view of an example of the modification of the coupling portion 103, B of FIG. 22 is a cross-sectional view illustrating how the coupling portion 103 of the modification illustrated in A of FIG. 22 is fitted into the modification illustrated in FIG. 19, and C of FIG. 22 is a perspective view illustrating a back surface of the coupling portion 103 of the modification illustrated in A of FIG. 22. In the present embodiment, the inlet 216 and/or the outlet 214 of the lid portion 213 for internal layer portion in the modification of the internal layer portion 211 that is illustrated in FIG. 19 may be sealed with a material exhibiting a high drug resistance and/or a less gas-permeable material until beginning of use (favorably, until just prior to usage) such that the internal layer portion 211 is hermetically sealed. Then, the material may be opened at the beginning of use by, for example, stabbing or puncture. Specifically, the following is an exemplary mechanism. The coupling portion 103 including a convex portion 1034 on a back surface of the lid portion 22, as illustrated in C of FIG. 22, is used to set for the lid portion 213 for internal layer portion. When the lid portion 22 is fitted into the coupling portion 103, the outlet 214 of the lid portion 213 for internal layer portion is opened with the convex portion 1034 by, for example, stabbing or puncture. Note that the less gas-permeable material and/or the less gas-permeable material has been already described above, and thus a description thereof is omitted here.

### (2-5) Others

A change in a capacity of the scent carrier 101 of the scent holding section 10 makes it possible to change, as necessary, an amount of scent to be held by the scent holding section 10. As a result, according to the amount of scent, there may be a plurality of types of scent holding sections 10 and different types of scent holding sections 10 may be included for the respective scent presentation modules 1. Further, the capacity of the scent carrier 101 is favorably changed without a size of the housing 13 or a joining portion of the housing 13 being affected. Specifically, the capacity is favorably changed in a longitudinal direction of the housing 13.

Further, the scent holding section 10 itself may be a scent holding section of a replaceable cartridge type, as described above, that is, the scent holding section 10 can also be replaced independently of the airflow forming section 11 and controller 12 described later. Thus, only the scent holding section 10 is disposable by a user when the scent holding section 10 no longer has scent. This makes it possible to improve usability and also to consider global environment.

### (2-6) Example of Scent Release Operation Performed by Scent Holding Section 10

FIG. 7 is a cross-sectional conceptual diagram illustrating how a scent is released from the scent holding section 10 of the present embodiment. An example of an operation of release of a scent-containing airflow that is performed by the scent holding section 10 is described below with reference to FIG. 7. Note that K in FIG. 7 indicates movement of airflow.

In the present embodiment, when the venting portion 102 is opened in a state in which a scent is held in the scent carrier 101, the air K flows into the venting portion 102 from the outside. The inflow air K flows into the coupling portion 103 and passes through one of coupling openings that is the coupling opening 40a, and the airflow K is delivered to a scent. The scent is added to the airflow K when the airflow K passes by the impregnation member 20, and a scent-containing airflow K is released. In this state, the released scent-containing airflow K flows into the coupling portion 103 through another of the coupling portions that is the coupling opening 40b, and accordingly, the scent-containing airflow K is released externally.

Specifically, in the present embodiment, the vent 30 (30a, 30b) includes an open-and-close mechanism that includes a sealing lid, a shaft, and a spring. Likewise, the coupling portion 103 also includes an open-and-close mechanism that includes a sealing lid, a shaft, and a spring. For example, a pushing portion X is inserted from a lower portion of a cartridge 10 on the side of, for example, the airflow forming section 11 to be coupled to the shaft. Accordingly, those open-and-close mechanisms can be controlled.

Note that FIG. 7 only illustrates a state in which the pushing portion X and the shaft are coupled to each other. When the pushing portion X is pushed in a state of being coupled to the shaft, the pushing portion X presses the first sealing lid 1021a inward of the first venting portion 102a. Then, the inflow vent 30a is opened, and the air K flows into the first venting portion 102a. Here, the first spring 1023a is contracted by the first sealing lid 1021a.

When the first sealing lid 1021a is pressed inward of the first venting portion 102a, the shaft 1022 mounted on the first sealing lid 1021a is moved in a direction of the coupling portion 103. The shaft 1022 presses a sealing lid 1031 inward of the coupling portion 103, and the air K in the first venting portion 102a flows into the coupling portion 103. Here, a spring 1033 is contracted by the sealing lid 1031.

Due to the presence of the partition portion 41, first, the air K flowing into the coupling portion 103 flows into the scent carrier 101 through the first coupling opening 40a provided to the lid portion 22, and is delivered to a scent held by the scent carrier 101. As a result, the scent is added to the airflow when the airflow passes by the impregnation member 20, and a scent-containing airflow K is released. The scent-containing airflow K flows into the coupling portion 103b again through the second coupling opening 40b provided to the lid portion 22.

When the sealing lid 1031 is pressed inward of the coupling portion 103, a shaft 1032 mounted on the sealing lid 1031 is moved in a direction of the second venting portion 102b. The shaft 1032 presses a second sealing lid 1021b inward of the second venting portion 102b, and a scent-containing air in the coupling portion 103 flows into the second venting portion 102b. Here, a second spring 1023b is contracted by the second sealing lid 1021b.

In the present embodiment, the second venting portion 102b communicates with the release vent 30b. Thus, the scent-containing airflow K flowing into the second venting portion 102b is released externally through the release vent 30b.

When the pushing of the pushing portion X is released after the scent-containing airflow K is released externally, the first sealing lid 1021a returns to its original state due to a restoring force of the contracted first spring 1023a. Further, the sealing lid 1031 returns to its original state due to a restoring force of the spring 1033, and the second sealing lid 1021b returns to its original state due to a restoring force of the second spring 1023b.

### (3) Airflow Forming Section 11

The airflow forming section 11 forms a scent-containing airflow, and releases the airflow externally through a release opening. The airflow forming section 11 includes the pushing portion X coupled to the shaft of the scent holding section 10, a drive source that drives the pushing portion X, and a drive source fixing portion that fixes the drive source. An SMA slider that folds a shape memory alloy SMA to slide the folded shape memory alloy is provided around a tip of the pushing portion X. The drive source fixing portion fixes the shaft of the scent holding section 10 and the drive source such that the pushing portion X is situated at a position at which the pushing portion X is coupled to the shaft.

Further, the entirety of the airflow forming section 11 is held in the housing 13 described later, and wiring through which power can be supplied is connected to the drive source. The drive source may be supplied with power through the wiring from the cell 14 (including a storage cell), such as a battery, that is stored in the housing 13, as illustrated in FIG. 3, or the drive source may be supplied with power through the wiring from a power supply provided outside of the housing 13. Note that a form of the cell 14 is not particularly limited, and can be designed discretionarily according to, for example, a form of the housing 13. Examples of the form of the cell 14 may include a substantially columnar form, a substantially conical form, a substantially cylindrical form, and a form of a substantially square column.

Due to extension and shrinkage of the shape memory alloy SMA, the pushing portion X is movable in the housing 13 in a direction in which the pushing portion X extends. The pushing portion X may be in any form that enables the pushing portion X to press the shaft of the scent holding section 10, and examples of the form of the pushing portion X may include a substantially columnar form, a substantially conical form, a substantially cylindrical form, and a form of a substantially square column.

The shape memory alloy SMA is folded into a U-shape by the SMA slider provided around the tip of the pushing portion X, and passes through the pushing portion X. Two ends of the shape memory alloy SMA are fixed to a rear end of the pushing portion X. In the present embodiment, an actuator that is the drive source is not limited to being formed using a shape memory alloy SMA. Any linear-movement mechanism, such as a motor, a solenoid, a linear sliding actuator, a pneumatic (air-pump) actuator, or a small electromagnetic actuator, that moves the pushing portion X linearly, may be adopted. Here, the linear-movement mechanism includes not only a mechanism of which a single member is moved linearly, but also a mechanism of which a portion of a plurality of coupled members are moved linearly.

Further, the airflow forming section 11 may include a blowing section (not illustrated) in addition to the pushing portion X, the drive source, and the drive source fixing portion. Examples of the blowing section include an axial flow fan, a blower fan, and a centrifugal fan. Further, alternatively, the blowing section may be a combination of a diaphragm pump and an airflow increasing mechanism, that is, the blowing section may especially be a combination of a diaphragm pump (such as a micro-blower) that uses a piezoelectric element or a motor, and an airflow increasing mechanism. The blowing section may be configured such that airflow formed using a diaphragm pump flows through a flow path after the airflow is increased by an airflow increasing mechanism.

Note that, when the blowing section is used, noise may be produced according to, for example, the types of blowing sections or the number of blowing sections mounted. For example, physical stimulation and/or chemical stimulation is given to liquid, the liquid is vaporized to expand a balloon, and gas is generated. Accordingly, the issue described above can be solved.

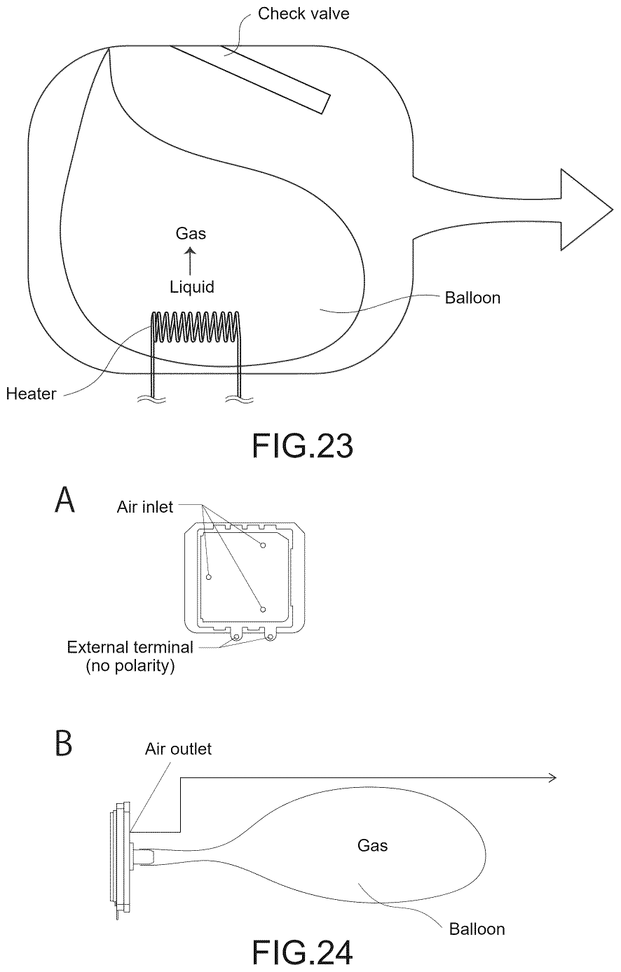

Specifically, thermal stimulation is given to liquid situated in a balloon using, for example, a heater, a thermistor, or a Peltier element, the liquid is vaporized to generate gas, a balloon is expanded, and the air is generated to be discharged, as illustrated in FIG. 23. Accordingly, the blowing source is silenced. Further, when a temperature in a balloon becomes high, the balloon remains expanded. Thus, there is no need to worry about continuously giving off a scent. This also makes it possible to, for example, prevent a scent from continuously being given off in a closed space such as in a vehicle.

Liquid that is thermally expanded easily is favorably adopted as the liquid in a balloon. Specific examples of the liquid in a balloon include alcohol such as methanol and ethanol, although the present technology is not particularly limited thereto. Further, a check valve may be provided in a balloon, as illustrated in FIG. 23.

There is another solution. For example, pressure is stored in a balloon in a state in which an operating voltage for a blowing source is relatively reduced, and the pressure is released upon air discharge, as illustrated in FIG. 24. Accordingly, a blowing source that produces reduced noise can also be obtained. Note that A of FIG. 24 schematically illustrates an example of an embodiment of a blowing source, as viewed from the front, and B of FIG. 24 schematically illustrates a back surface of A of FIG. 24. Further, on the same principle as the description above, the noise reduction can be achieved using a syringe movement performed by a linear-movement actuator. Furthermore, a check valve (not illustrated) may also be provided to a balloon illustrated in B of FIG. 24.

Moreover, for example, liquid is vaporized instantly in a metallic piston by discharge (such as spark discharge, DC arc discharge, or glow discharge), and the air is discharged instantly. Accordingly, the blowing source is silenced.

### (4) Modification of Airflow Forming Section 11

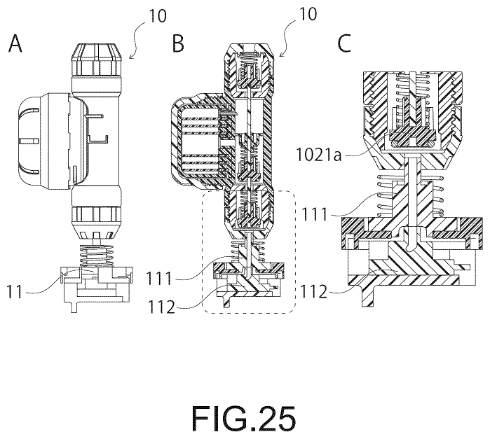

FIG. 25 illustrates an example of a modification of the airflow forming section 11. A of FIG. 25 is a front view, B of FIG. 25 is a cross-sectional view, and C of FIG. 25 is an enlarged view of a portion of B of FIG. 25. In the modification illustrated in FIG. 25, the airflow forming section 11 includes a pump 111 that presses the shaft of the scent holding section 10, and a movable portion 112 that moves the pump. Note that the pump 111 in the modification is a pump having a larger area of contact with the scent holding section 10 (especially the first sealing lid 1021a) than the pushing portion X described above. In other words, favorably, the pump 111 is not coupled to the shaft of the scent holding section 10.

In the modification, the entirety of the airflow forming section 1 may be held in the housing 13 described later, and wiring through which power can be supplied is connected to the drive source. The drive source may be supplied with power through the wiring from the cell 14 (including a storage cell), such as a battery, that is stored in the housing 13, or the drive source may be supplied with power through the wiring from a power supply provided outside of the housing 13.

In the modification, a tip of the pump 111 is not coupled to the shaft, but is brought into contact with the first sealing lid 1021a including the shaft. This results in opening the first sealing lid 1021a. This makes it possible to reduce the number of parts, and thus to make the airflow forming section 11 smaller in size. Further, airflow can be formed efficiently.

The pump 111 may be in any form that enables the tip to be brought into contact with the first sealing lid 1021a, and, for example, the pump 111 may be in a substantially columnar form, in a substantially conical form, in a substantially cylindrical form, in a form of a substantially square column, or in a form obtained by combining at least one thereof. Further, for example, the pump 111 may be in a form of combined columns of different sizes. Furthermore, the pump 111 may include, for example, a return spring as necessary, as illustrated in FIG. 25.

Any movable portion that can move the pump in a linear-movement direction may be used as the movable portion 112. Examples of the movable portion 112 include an actuator formed using a shape memory alloy SMA, a motor, a solenoid, a linear sliding actuator, a pneumatic (air-pump) actuator, and an actuator including a small electromagnet or the like. Here, a linear-movement mechanism includes not only a mechanism of which a single member is moved linearly, but also a mechanism of which a portion of a plurality of coupled members are moved linearly.

### (5) Controller 12

The controller 12 controls an operation of the airflow forming section 11. The controller 12 may be provided at any position that enables the controller 12 to control the operation of the airflow forming section 11, and may be provided anywhere in the housing 13 described later. For example, the controller 12 may be provided at a position in a longitudinal direction of the drive source fixing portion.

For example, the controller 12 controls at least one of a distance by which a scent is presented, a range in which the scent is presented, a direction in which the scent is released, a period of time for which the scent is released, or a concentration of the presented scent, and performs control on the airflow forming section 11 such that the airflow forming section 11 releases the scent. This control can be performed by changing, for example, a speed at which the pushing portion X or the pump can be moved, a period of time for which the pushing portion X or the pump can be moved, or the number of times that the pushing portion X or the pump can be moved. For example, an instruction is given to the controller 12 through a wired or wireless network using a linked terminal. Specifically, the terminal is linked to an application (such as a native app, a Web app, a hybrid app, and a built-in app), and the control is performed in accordance with a program in the application.

### (6) Housing 13

The housing 13 includes therein at least the scent holding section 10, the airflow forming section 11, and the controller 12. Further, in the present embodiment, the housing 13 includes a joining portion that enables the housing 13 to be joined to another housing.

Any material may be adopted as a material used to form the housing 13. For example, metal such as stainless steel (steel use stainless (SUS)) or aluminum; resin such as polyvinyl chloride, polyethylene, phenol resin, olefin resin, acrylic resin, urethane resin, ABS resin, polyether ether ketone (PEEK) resin, polyacetal (POM) resin, fluororesin, cycloolefin polymer resin, or polyimide resin; earthenware; porcelain; or ceramics may be used as the material used to form the housing 13, or at least one thereof may be combined discretionarily to be used as the material used to form the housing 13.

The housing 13 may also be in any form that enables the housing 13 to include therein at least the scent holding section 10, the airflow forming section 11, and the controller 12, and can be designed discretionarily. For example, a substantially cylindrical form, a substantially polygonal cylindrical form having a substantially polygonal cross section (a substantially triangular or rectangular cross section, or a cross section with five or more sides), a substantially conical form, or a substantially polygonal conical form having a substantially polygonal cross section (a substantially triangular or rectangular cross section, or a cross section with five or more sides) may be used as the form of the housing 13, or at least one thereof may be combined discretionarily to be used as the form of the housing 13.

In the present embodiment, the housing 13 includes the joining portion (not illustrated), as described above. Favorably, the other housing is the housing 13 including therein the scent holding section 10, the airflow forming section 11, and the controller 12. In other words, the other housing corresponds to another scent presentation module 1. Any number may be adopted as the number of other housings joined, and the housing 13 can be joined to at least one other housing as necessary.

Favorably, the joining portion is formed such that housings are removable from each other as necessary. Specifically, the joining portion can be formed by providing, inside or outside of the housing 13, a material that includes a magnetic member. Moreover, the joining portion can be formed by providing, on an outer surface of the housing 13, a fitting mechanism used to fit the housings 13. The following is an example of the fitting mechanism. An uneven form is provided on the outer surface of the housing 13, and the uneven forms fit each other. Further, the joining portion may also be formed by performing threading on the outer surface of the housing 13 (a male screw or a female screw); by including a cam latch handle or a lock part having, for example, a buckle structure; or by using a bonding layer having adhesive properties that enables the bonding layer to be removable. The outer surface may be one of, for example, an upper surface of, a bottom surface of, a lateral side surface of, and a longitudinal side surface of the housing 13.

### (7) Connection With External Unit

The scent presentation module 1 according to the present embodiment can be connected to an external unit through a wired or wireless network. In the present embodiment, the establishment of connection with an external unit makes it possible to operate the external unit at any timing.

Any unit may be used as the external unit connectable to the scent presentation module 1. For example, a fan unit that generates airflow, a power supply unit that supplies power, a mixing unit, a sensing unit, a deodorization unit that performs deodorization (the case in which the deodorization unit is arranged distant from the scent presentation module 1 is also included), an airflow control unit, a blowing unit that generates airflow in a deodorization direction, a device for performing control on pose maintenance, a storage unit that stores therein information related to the scent presentation module 1, or a charge unit may be used as the external unit, or at least one thereof may be combined discretionarily to be used as the external unit.

For example, the sensing unit performs not only biological sensing including sensing related to, for example, human detection, a body temperature, a heart rate, a sweat volume, and face recognition, but also surrounding-environment-related sensing including sensing related to, for example, a temperature, humidity, brightness, a distance, and a scent. Further, any device that can fix a position of the scent presentation module 1 may be used as the device for performing control on pose maintenance, and examples of the device include a stand, a clip, a tripod, and a hanging mechanism. Furthermore, the examples of the device may be formed using, for example, a bar or a plate member, or may be formed using, for example, a shape memory material.

For example, the airflow control unit may be an airflow control mechanism that is provided around a release opening of the scent presentation module 1 and that controls a release direction of and/or a release strength of the airflow released through the release opening. Specific examples of the airflow control unit include a louvre that controls a direction in which airflow is released and a wind direction adjusting structure. Further, the release direction of and/or the release strength of the airflow may be controlled by creating a wall using ultrasonic waves and by only releasing a scent-containing airflow to a certain portion.

The mixing unit is used to mix a plurality of scents as necessary when a scent presentation system 2 described later includes a plurality of scent presentation modules 1.

The scent presentation module 1 according to the present embodiment may further include a communication section (not illustrated) that is wirelessly connected to an external unit when connection with the external unit is established wirelessly. In this case, the communication section may be wirelessly connected to an external unit using a communication approach such as Bluetooth (registered trademark), Wi-Fi, or SIM communication. However, the present technology is not limited to these communication approaches.

### (8) Information Related to Scent Presentation Module 1

The scent presentation module 1 according to the present embodiment can hold information related to the scent presentation module 1. Examples of the information related to the scent presentation module 1 include an ID of the scent presentation module 1, a position of the scent presentation module 1, a history of past use of the scent presentation module 1, a date of manufacturing of the scent presentation module 1, a place of manufacturing of the scent presentation module 1, whether a scent has been released, a period of time for which a scent is released, the type of scent, a concentration of a scent, a remaining amount of scent, an expiration date of scent, a date of manufacturing of a scent, a place of manufacturing of a scent, and an ID of the mounted scent holding section 10. However, the present technology is not limited to these pieces of information. Note that the scent holding section 10 may refer to a database on the Web to search for information regarding EEPROM and an ID of the scent holding section 10, and may download information (such as scent information) registered with the database to record therein the information.

A storage (not illustrated) may be provided in the scent presentation module 1 and the information related to the scent presentation module 1 may be stored in the storage, or connection may be established with an external storage unit through a wired or wireless network and the information may be stored in the storage unit.

The information related to the scent presentation module 1 may be fed back to at least one external unit described above through a wireless or wired network. When the information related to the scent presentation module 1 is fed back to the external unit, for example, a concentration of a scent is adjusted, a direction in which a scent is released is adjusted, an alert that gives an instruction to replace the scent holding section 10 or the scent presentation module 1 is put out, presentation of a scent that is performed by the scent presentation module 1 is started, stopped, or finished. However, the present technology is not particularly limited thereto.

Further, not only is control performed on the scent holding section 10 or the scent presentation module 1 due to the feedback described above, the scent holding section 10 or the scent presentation module 1 may work with an external unit, and the scent holding section 10 or the scent presentation module 1 and the external unit may be controlled at a time due to the feedback described above. For example, adjustment of a concentration of a scent by the scent presentation module 1 and control on a release direction of and/or release strength of the airflow by an airflow control unit can be performed at a time.

### (9) Air Passage 15

The scent presentation module 1 according to the present embodiment may further include an air passage 15 that circulates the air from one of two ends of the scent presentation module 1 to another of the two ends. The inclusion of the air passage 15 enables airflow to also pass through the scent presentation module 1. For example, a scent-containing airflow can be more efficiently delivered using, for example, an auxiliary airflow.

Specifically, the air passage 15 is formed continuously from one of two ends of the scent presentation module 1 to another of the two ends in a longitudinal direction of the scent presentation module 1. The housing 13 stores therein the scent holding section 10, the airflow forming section 11, and the controller 12, and these structural elements are situated around the air passage 15. In other words, the air passage 15 is formed to avoid these structural elements.

### (10) Others

An outer surface of the scent presentation module 1 may be marked using, for example, a marker or an LED light, and a housing colored in advance may be used. When the scent presentation module 1 may be marked using an LED light, there may be a temporal change in the color of the scent presentation module 1. The marking may be associated with information such as a scent held by the scent presentation module 1, a concentration of a scent, a date of manufacturing of a scent, a remaining amount of scent, and a remaining life of a battery. Further, the marking may be performed to just provide a design quality without being associated with these pieces of information.

The marking may be performed at any position situated on the outer surface of the scent presentation module 1, and examples of the position include an upper surface, a bottom surface, a lateral side surface, and a longitudinal side surface. Favorably, marking may be performed longitudinally on the longitudinal side surface.

### 2. Second Embodiment (Scent Presentation System 2)

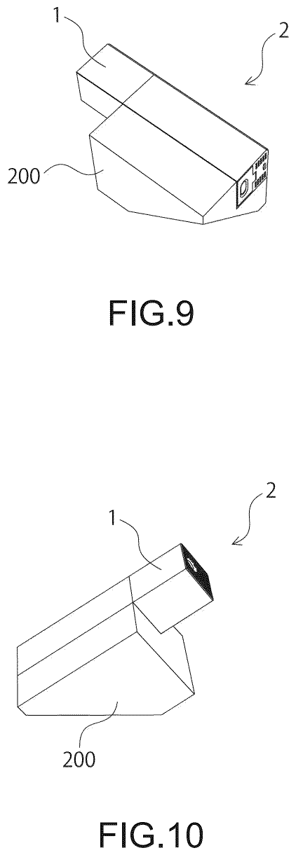

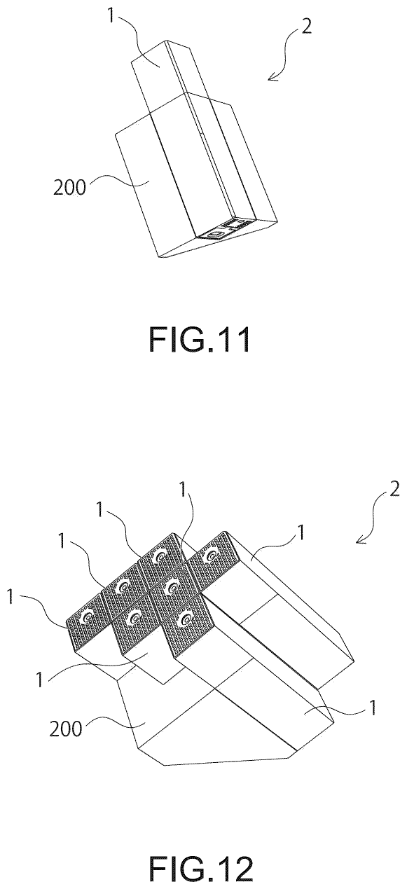

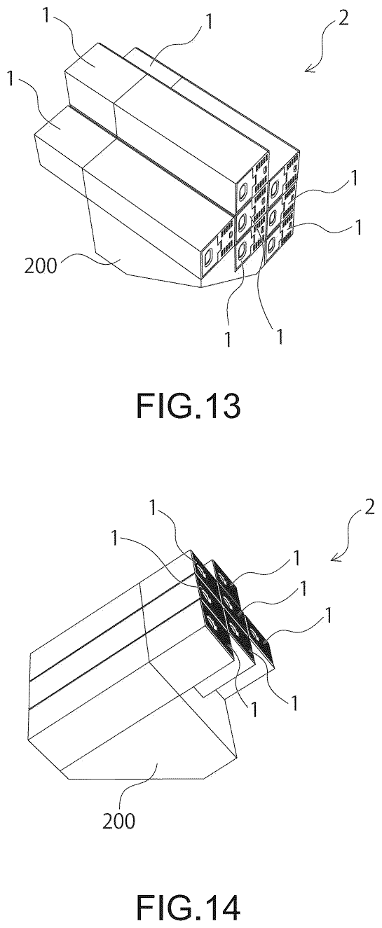

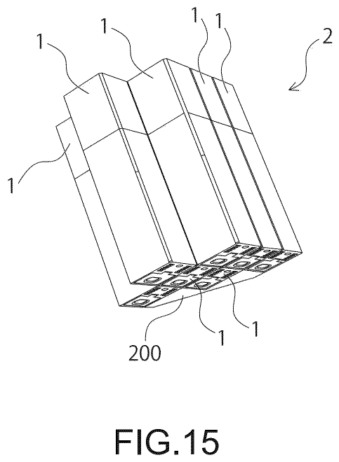

An example of a configuration of the scent presentation system 2 according to a second embodiment of the present technology is described with reference to FIGS. 8 to 15. FIGS. 8 to 11 are perspective views of the scent presentation system 2 according to the present embodiment, as viewed from various angles, when the scent presentation system 2 includes a single scent presentation module 1. FIGS. 12 to 15 are perspective views of the scent presentation system 2 according to the present embodiment, as viewed from various angles, when the scent presentation system 2 includes a plurality of scent presentation modules 1.

The scent presentation system 2 according to the present embodiment includes at least one scent presentation module 1 described above, and presents a scent using any of the at least one scent presentation module 1. In this case, the number of any of the at least one scent presentation module 1 may be determined as necessary.

The scent presentation system 2 according to the present embodiment is described in detail below.

The present embodiment also includes the case in which the scent presentation modules 1 are joined to each other, as illustrated in FIGS. 12 to 15. For example, the joining is performed using a joining portion of the scent presentation module 1. For example, the scent presentation modules 1 may be joined in a grid in, for example, a top direction, an X direction, a Y direction, a Z direction, or all of directions obtained by combining at least one thereof discretionarily. The number of scent presentation modules 1 joined is also not particularly limited. For example, the scent presentation modules 1 are provided obliquely to a horizontal plane to be joined in a grid in FIGS. 12 to 15.

Further, the scent presentation system 2 may include, for example, a base 200 used to fix the scent presentation module 1 in a desired direction such as fixing the scent presentation module 1 obliquely to a horizontal plane, as illustrated in FIGS. 8 to 15.

When the scent presentation system 2 includes at least one scent presentation module 1, this makes it possible to provide expandability and flexibility in the number of scent presentation modules 1, arrangement of the scent presentation module 1, and control on the scent presentation module 1. This makes it possible not only to release a scent to the entirety of a space but also to give off a scent only in a small cubic or spherical space. This can be achieved by using the scent presentation modules 1 independent of each other, by scattering the scent presentation modules 1 as necessary, by operating the scent presentation modules 1 in an integral or independent manner, and by only releasing a scent from at least one specific scent presentation module 1.

Further, the number of scent presentation modules 1 can be increased or decreased discretionarily. Thus, a necessary number of scents to be presented can be provided discretionarily. Furthermore, the scent presentation modules 1 can be controlled independently, as described later. This results in also providing an advantage in providing a high degree of time responsiveness.

In the present embodiment, the scent presentation modules 1 of the scent presentation system 2 are joined to each other and can be controlled independently of each other. Specifically, the scent presentation system 2 controls each scent presentation module 1 by being connected to the controller 12 of the scent presentation module 1 through a wired or wireless network and by transmitting a drive signal to the controller 12. In the present embodiment, the scent presentation modules 1 each include the controller 12. Thus, the scent presentation modules 1 can be controlled independently. This makes it possible to control discretionarily combined scent presentation modules 1 at a time. In other words, the present technology can provide not only a flexible design, but also a unified design in control mechanism and a unified design in control management.

For example, the drive signal is transmitted through a wired or wireless network using a terminal linked to an external unit such as the storage unit described above or to the scent presentation module 1. For example, the drive signals are transmitted at a time through a wired or wireless network using a linked terminal. Specifically, the terminal is linked to an application, and the control is performed in accordance with a program in the application. Note that of course partial control can also be performed when there is no need to perform control at a time in the present technology.

Note that any combination may be adopted as the combination of the scent presentation modules 1, and, for example, the scent presentation modules 1 of different scents or the scent presentation modules 1 of different scent concentrations may be joined.

Further, when the scent presentation module 1 further includes an air passage that circulates the air from one of two ends of the scent presentation module 1 to another of the two ends in the present embodiment, the scent presentation system may supply the air through the one of the two ends to generate an auxiliary airflow that assists the scent-containing airflow. This makes it possible to deliver a scent-containing airflow efficiently in a direction in which the scent-containing airflow is desired to be delivered. Specifically, it is conceivable that, for example, a fan could be provided in the scent presentation system 2 and started in response to a scent being presented by the scent presentation module 1, or presentation of a scent by the scent presentation module 1 could be tied to a fan unit that is connected to the scent presentation system 2 through a wired or wireless network. However, the present technology is not particularly limited thereto. Further, an installation place for a fan unit in this case is not particularly limited, and the fun unit can be installed at any place according to, for example, a distance by which a scent is desired to be presented.

The scent presentation system 2 may be connected to at least one auxiliary airflow generating module (not illustrated) through a wired or wireless network, and may generate an auxiliary airflow at any timing using the auxiliary airflow generating module. Examples of the auxiliary airflow generating module include a small fan, although the present technology is not particularly limited thereto. Further, a housing of the auxiliary airflow generating module may be in a form similar or analogous to the form of the scent presentation module 1. In this case, the auxiliary airflow generating module can be joined to the scent presentation module 1 or the auxiliary airflow generating module. With respect to joining, a joining portion is favorably formed such that housings are removable from each other as necessary, as in the case of the joining portion of the housing 13. Specifically, the joining portion can be formed by providing, inside or outside of the housing of the auxiliary airflow generating module, a material that includes a magnetic member. Moreover, the joining portion can be formed by providing, on an outer surface of the housing, a fitting mechanism used to fit the housings.

As in the case of the scent presentation module 1, the scent presentation system 2 can be connected to an external unit through a wired or wireless network. In this case, the scent presentation modules 1 in the scent presentation system 2 may be connected to the external unit independently of each other, a portion of the scent presentation modules 1 may be connected to the external unit together, or all of the scent presentation modules 1 included in the scent presentation system 2 may be connected to the external unit at a time.

Any external unit may be adopted as the external unit connectable to the scent presentation system 2, and examples of the external unit are similar to those described in “(7) Connection with External Unit” of “1. First Embodiment (Scent Presentation Module 1).” Thus, a description thereof is omitted. From among the external units described above, the scent presentation system 2 is connected to the deodorization unit through a wired or wireless network, and can perform deodorization operation at any timing (such as a timing after the scent presentation module 1 releases a scent-containing airflow) using the deodorization unit. The timing may be determined on the basis of information fed back through a wired or wireless network.

In this case, any place may be adopted as an installation place for the deodorization unit, and the deodorization unit may be installed before or after the scent presentation system 2 or at a position distant from the scent presentation system 2 by a few centimeters or a few meters. Examples of the deodorization unit include a conventionally known blowing apparatus, and a mechanism that is moved in an up-and-down direction, in a right-and-left direction, or in a rotation direction due to airflow or using a power source such as an actuator, although the present technology is not particularly limited thereto.

Further, the scent presentation system 2 is connected to a charge unit through a wired or wireless network, and can charge at least one scent presentation module at a time using the charge unit. This makes it possible to provide a system with a high degree of usability, where the scent presentation modules 1 can be controlled independently of each other and at least one scent presentation module 1 can be charged at a time. Examples of the charge unit include a conventionally known charger (also including a contact or contactless charger), although the present technology is not particularly limited thereto.

Further, the scent presentation system 2 is connected to at least one fan unit through a wired or wireless network, and a distance by which a scent is presented can be controlled using the fan unit. In other words, the distance by which a scent is presented can be changed between a short distance (for example, from 15 to 30 centimeters) and a medium distance (from one to three meters). Examples of the fan unit include a conventionally known blowing apparatus, and various blowing apparatuses may be combined to be used according to the distance by which a scent is presented. However, the present technology is not particularly limited thereto. Further, when the type of blowing section included in the airflow forming section 11 of the scent presentation module 1 is changed as appropriate or the blowing sections are combined as appropriate, this makes it possible to control the distance by which a scent is presented, without using an external unit.

Further, in the present technology, what is referred to as external units in the description above may be modularized, as in the case of the auxiliary airflow generating module described above, and a housing of a module obtained by the modularization may be configured to be joinable to the scent presentation module 1 or the module obtained by the modularization. With respect to joining, a joining portion is favorably formed such that housings are removable from each other as necessary, as in the case of the joining portion of the housing 13. Specifically, the joining portion can be formed by providing, inside or outside of the housing of the scent presentation module 1, a material that includes a magnetic member. Moreover, the joining portion can be formed by providing, on an outer surface of the housing, a fitting mechanism used to fit the housings. Further, in this case, the other scent presentation modules 1 may also be controlled at a time through a wired or wireless network using a linked terminal.

Further, in the present technology, the scent presentation modules 1 included in the scent presentation system 2 can be connected to each other through a wired or wireless network. Specifically, for example, a portion of or all of the scent presentation modules 1 establish near-field connection with each other (such as wireless magnetic communication using, for example, a magnet in the housing 13), and this makes it possible to provide a control signal to all of the presentation modules 1.

### 3. Third Embodiment (Information Processing Apparatus)

An information processing apparatus according to the present embodiment includes a terminal that is linked to a scent presentation module 1 that includes a scent holding section 10 that holds a scent, an airflow forming section 11 that forms a scent-containing airflow and that releases the airflow externally through a release opening of the scent presentation module 1, a controller 12 that controls an operation of the airflow forming section 11, and a housing 13 that includes therein the scent holding section 10, the airflow forming section 11, and the controller 12, the housing 13 including a joining portion that enables the housing 13 to be joined to another housing 13; and a processor that receives information that is related to a state of the scent presentation module 1 and available to a user, that performs processing of outputting and storing the information, and that generates a signal related to the output and storing related to the information.

The information processing apparatus according to the present embodiment is described in detail below.

In the present embodiment, the terminal is linked to an application that controls the scent presentation module 1, and examples of the application include a native app, a Web app, a hybrid app, and a built-in app. Examples of the terminal include a laptop, a desktop, a computer, a tablet, a smartphone, a personal digital assistant, a game machine, and a television. However, the present technology is not particularly limited thereto.

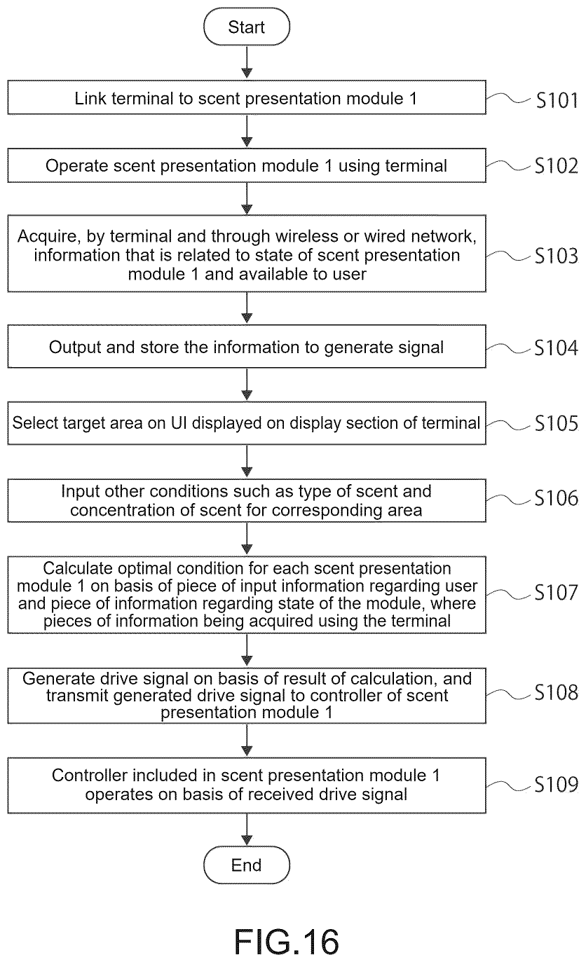

Processing performed using the information processing apparatus according to the present embodiment is described using a flowchart illustrated in FIG. 16.

First, a terminal is linked to the scent presentation module 1 (the number is not particularly limited.) (S101). In S101, the terminal may be linked to not only the scent presentation module 1 but also the external unit described above and another scent presentation module 1. Next, the scent presentation module 1 is operated through a wired or wireless network using the terminal (S102). This operation makes it possible not only to turn on and off the scent presentation module 1 and operate the controller 12 of the scent presentation module 1 (also including control on at least one of the above-described distance by which a scent is presented, the above-described range in which the scent is presented, the above-described direction in which the scent is released, the above-described period of time for which the scent is released, or the above-described concentration of the presented scent, but also to operate the external unit described above and another scent presentation module 1 at a time.

Next, in the information apparatus according to the present embodiment, the terminal linked to the scent presentation module 1 acquires, through a wireless or wired network, information that is related to a state of the scent presentation module 1 and available to a user (S103). Here, the acquired information is output and stored by a processor of the present embodiment to generate a signal related to the output and storing related to the information in order to transmit the signal to the network (S104). Here, information other than the information related to the state of the scent presentation module 1 and available to a user may also be acquired at the same time. Examples of the information related to the state of the scent presentation module 1 and available to a user include a position of the scent presentation module 1 (also including, for example, an installation position and an installation space in concept), the type of scent, a concentration of the scent, a remaining amount of the scent, and a period of time for which the scent is released.

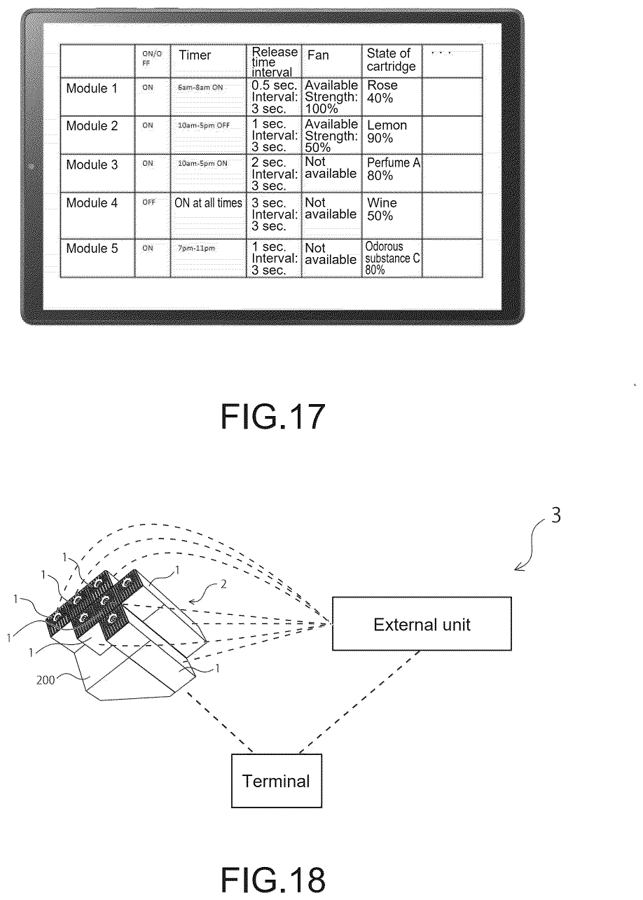

Next, for example, a target area is selected on a UI (refer to FIG. 17) displayed on a display section of the terminal (S105). Specifically, it is conceivable that any block could be selected from a screen on which a space is partitioned in a grid or any block could be specified from among not rectangular but spherical blocks provided in a space. However, the present technology is not particularly limited thereto. Then, conditions for a space represented by the selected block are input to the terminal using the UI displayed on the display section of the terminal (S106), where examples of the conditions include the type of a desired scent, a concentration of the scent, a remaining amount of the scent, and a period of time for which the scent is released. Note that, in the present embodiment, the processes of S105 and S106 are performed in a random order, and the process of S105 may be performed after the process of S106.

Next, an optimal condition for each scent presentation module 1 is calculated on the basis of a piece of input information regarding a user and the piece of information related to the state of the presentation module 1, the pieces of information being acquired using the terminal (S107). In the present embodiment, the use of the two pieces of information makes it possible to obtain the optimal condition for the scent presentation module 1 with a high degree of accuracy. Further, a drive signal is generated on the basis of a result of the calculation and transmitted to the controller 12 of the scent presentation module 1 (S108). Furthermore, a drive signal other than the drive signal may also be generated, and the generated other drive signal may be transmitted to the external unit described above or another scent presentation module 1 at a similar timing.

In the scent presentation module 1, the controller 12 included in the scent presentation module 1 operates on the basis of the received drive signal, and a cent-containing airflow is released (S109). Further, when a scent-containing airflow is released, a report indicating that the scent-containing airflow has been released may be output to not only the controller 12 of the scent presentation module 1 but also the external unit described above or another scent presentation module 1, although this is not indicated by the flowchart in FIG. 16. This makes it possible to, for example, start a deodorization unit or generate an auxiliary airflow when a scent has not been delivered.

### 4. Fourth Embodiment (Scent Presentation Module Controlling System 3)

FIG. 18 illustrates an example of a configuration of a scent presentation module controlling system 3 according to a fourth embodiment of the present technology. The scent presentation module controlling system 3 according to the present embodiment includes at least one scent presentation module 1; a terminal that is linked to the scent presentation module 1; and an information processing apparatus including a processor that receives information related to a state of the module and available to a user, that performs processing of outputting and storing the information, and that generates a signal related to the output and storing related to the information. Note that, in FIG. 18, the information processing apparatus is illustrated as an “external unit” since the information processing apparatus is an example of the external unit.

The “at least one scent presentation module 1”, the “terminal”, and the “information processing apparatus” are similar to those described in “1. First Embodiment (Scent Presentation Module 1)” to “3. Third Embodiment (Information Processing Apparatus).” Thus, descriptions thereof are omitted. Note that all of the presentation modules 1 are connected to the external unit in FIG. 18. However, the present technology is not limited thereto, and one of or a limited portion of the scent presentation modules 1 may be connected to the external unit. Further, a portion of or all of the scent presentation modules 1 establish near-field connection with each other, and this makes it possible to provide a control signal to all of the presentation modules 1, as described above.

### 5. Application of Scent Presentation Module 1 and Scent Presentation System 2

The scent presentation module 1 and the scent presentation system 2 are used for, for example, an olfactory test or an olfactory training (an olfactory stimulation therapy). Note that the olfactory training as used herein is broadly construed, and may include trainings for, for example, an exam of olfactory measurement operators, exams of sommeliers, and the Aromatherapy Certification. Further, the scent presentation module 1 and the scent presentation system 2 may be used for flavor simulation performed upon developing food-and-drink products. Further, the scent presentation module 1 and the scent presentation system 2 may be included in an automobile, a head-mounted display, or a relaxing product such as a neck pillow or an eye pillow. Furthermore, the scent presentation module 1 and the scent presentation system 2 may be included in, for example, a customer-attracting product such as a vending machine, a digital signage, or a robot.

Note that the present technology may adopt the following configurations.
[1] A scent presentation module, including:
   a scent holding section that holds a scent;
   an airflow forming section that forms a scent-containing airflow and that releases the airflow externally through a release opening of the scent presentation module;
   a controller that controls an operation of the airflow forming section; and
   a housing that includes therein the scent holding section, the airflow forming section, and the controller, the housing including a joining portion that enables the housing to be joined to another housing.
[2] The scent presentation module according to [1], in which
   the joining portion is removable.
[3] The scent presentation module according to [2], in which
   the joining portion includes a magnetic member and/or includes a fitting mechanism used to fit the housings.
[4] The scent presentation module according to any one of [1] to [3], in which
   the controller controls at least one of a distance by which the scent is presented, a range in which the scent is presented, a direction in which the scent is released, a period of time for which the scent is released, or a concentration of the presented scent.
[5] The scent presentation module according to any one of [1] to [4], in which
   the scent presentation module is connectable to an external unit.
[6] The scent presentation module according to [5], further including
   a communication section that is wirelessly connected to the external unit.
[7] The scent presentation module according to [5] or [6], in which
   an airflow control mechanism that controls a release direction of and/or a release strength of the airflow released through the release opening of the module is provided around the release opening.
[8] The scent presentation module according to any one of [5] to [7], in which
   the scent presentation module holds information regarding the module.
[9] The scent presentation module according to [8], in which
   the information is fed back to the external unit.
[10] The scent presentation module according to any one of [1] to [9], further including
   an air passage that circulates air from one of two ends of the scent presentation module to another of the two ends.
[11] The scent presentation module according to any one of [1] to [10], in which
   the scent holding section further includes
   an internal layer portion that includes therein an impregnation member that carries the scent, and
   a lid portion for internal layer portion that corresponds to the internal layer portion, and
   a portion of or all of the internal layer portion and lid portion for internal layer portion is formed using a material exhibiting a high drug resistance and/or a less gas-permeable material.
[12] The scent presentation module according to any one of [1] to [11], in which
   the airflow forming section further includes a silenced blowing source or a blowing source that produces reduced noise.
[13] The scent presentation module according to any one of [1] to [12], in which
   the airflow forming section causes air to flow into the scent holding section by linearly moving a pushing portion or a pump having a larger area of contact with the scent holding section than the pushing portion.
[14] The scent presentation module according to [11], in which
   the material exhibiting a high drug resistance and/or the less gas-permeable material is at least one of stainless steel (steel use stainless (SUS)), aluminum, polytetrafluoroethylene (PTFE), ethylene tetrafluoroethylene (ETFE), or glass.
[15] The scent presentation module according to [12], in which
   the silenced blowing source is a mechanism that uses vaporization of liquid situated in a balloon.
[16] The scent presentation module according to [12], in which
   The blowing source producing reduced noise is a mechanism that uses storage of pressure in a balloon in a state in which an operating voltage for the blowing source is relatively reduced.
[17] A scent presentation system, including
   at least one scent presentation module that includes
      a scent holding section that holds a scent,
      an airflow forming section that forms a scent-containing airflow and that releases the airflow externally through a release opening of the scent presentation module,
      a controller that controls an operation of the airflow forming section, and
      a housing that includes therein the scent holding section, the airflow forming section, and the controller, the housing including, on an outer surface of the housing, a joining portion that enables the housing to be joined to another housing,
   the scent presentation system presenting the scent using any of the at least one scent presentation module.
[18] The scent presentation system according to [17], in which
   when a plurality of the modules is included, the modules are joined to each other.
[19] The scent presentation system according to claim or [18], in which
   the at least one scent presentation module is independently controllable.
[20] The scent presentation system according to any one of claims [17] to [19], in which
   the module further includes an air passage that circulates air from one of two ends of the module to another of the two ends, and
   the scent presentation system supplies the air through the one of the two ends to generate an auxiliary airflow that assists the scent-containing airflow.
[21] The scent presentation system according to any one of [17] to [20], in which
   the scent presentation system is connected to at least one auxiliary airflow generating module, and generates an auxiliary airflow at any timing using the at least one auxiliary airflow generating module.
[22] The scent presentation system according to [21], in which
   the auxiliary airflow generating module is connectable to the module.
[23] The scent presentation system according to any one of [17] to [22], in which
   the scent presentation system is connected to a deodorization unit, and performs deodorization operation at any timing using the deodorization unit.
[24] The scent presentation system according to any one of [17] to [23], in which
   the scent presentation system is connected to a charge unit, and charges the at least one scent presentation module at a time using the charge unit.
[25] An information management apparatus, including:
   a terminal that is linked to a scent presentation module that includes a scent holding section that holds a scent, an airflow forming section that forms a scent-containing airflow and that releases the airflow externally through a release opening of the scent presentation module, a controller that controls an operation of the airflow forming section, and a housing that includes therein the scent holding section, the airflow forming section, and the controller, the housing including a joining portion that enables the housing to be joined to another housing; and
   a processor
      that receives information that is related to a state of the module and available to a user,
      that performs processing of outputting and storing the information, and
      that generates a signal related to the output and storing related to the information.
[26] The information processing apparatus according to [25], in which
   the information processing apparatus
      calculates an optimal condition for each scent presentation module on the basis of a piece of input information regarding the user and the piece of information related to the state of the module, the pieces of information being acquired using the terminal,
      generates a drive signal on the basis of a result of the calculation, and
      transmits the drive signal to the controller of the module.
[27] A scent presentation module controlling system, including:
   at least one scent presentation module that includes
      a scent holding section that holds a scent,
      an airflow forming section that forms a scent-containing airflow and that releases the airflow externally through a release opening of the scent presentation module,
      a controller that controls an operation of the airflow forming section, and
      a housing that includes therein the scent holding section, the airflow forming section, and the controller, the housing including a joining portion that enables the housing to be joined to another housing;
   a terminal that is linked to the at least one scent presentation module; and
   an information processing apparatus including a processor that receives information that is related to a state of the module and available to a user, that performs processing of outputting and storing the information, and that generates a signal related to the output and storing related to the information.

### REFERENCE SIGNS LIST

1 scent presentation module
10 scent holding section
101 scent carrier
102 venting portion
102a first venting portion
102b second venting portion
103 coupling portion
1034 convex portion
20 impregnation member
21 container
211 internal layer portion
212 external layer portion
213 lid portion for internal layer portion
214 inlet
215 groove
216 outlet
30, 30a, 30b vent
31 internal layer member
32 external layer member
40, 40a, 40b coupling opening
41 partition portion
50 sealing container
51 container portion
52 lid portion for sealing container
11 airflow forming section
111 pump
112 movable portion
12 controller
13 housing
14 cell
15 air passage
2 scent presentation system
200 base
3 scent presentation module controlling system
K air, airflow

1. A scent presentation module, comprising:
a scent holding section that holds a scent;
an airflow forming section that forms a scent-containing airflow and that releases the airflow externally through a release opening of the scent presentation module;
a controller that controls an operation of the airflow forming section; and
a housing that includes therein the scent holding section, the airflow forming section, and the controller, the housing including a joining portion that enables the housing to be joined to another housing.

2. The scent presentation module according to claim 1, wherein
the joining portion is removable.

3. The scent presentation module according to claim 2, wherein
the joining portion includes a magnetic member and/or includes a fitting mechanism used to fit the housings.

4. The scent presentation module according to claim 1, wherein
the controller controls at least one of a distance by which the scent is presented, a range in which the scent is presented, a direction in which the scent is released, a period of time for which the scent is released, or a concentration of the presented scent.

5. The scent presentation module according to claim 1, wherein
the scent presentation module is connectable to an external unit.

6. The scent presentation module according to claim 5, further comprising
a communication section that is wirelessly connected to the external unit.

7. The scent presentation module according to claim 5, wherein
an airflow control mechanism that controls a release direction of and/or a release strength of the airflow released through the release opening of the module is provided around the release opening.

8. The scent presentation module according to claim 5, wherein
the scent presentation module holds information regarding the module.

9. The scent presentation module according to claim 8, wherein
the information is fed back to the external unit.

10. The scent presentation module according to claim 1, further comprising
an air passage that circulates air from one of two ends of the scent presentation module to another of the two ends.

11. The scent presentation module according to claim 1, wherein
the scent holding section further includes
an internal layer portion that includes therein an impregnation member that carries the scent, and
a lid portion for internal layer portion that corresponds to the internal layer portion, and
a portion of or all of the internal layer portion and lid portion for internal layer portion is formed using a material exhibiting a high drug resistance and/or a less gas-permeable material.

12. The scent presentation module according to claim 1, wherein
the airflow forming section further includes a silenced blowing source or a blowing source that produces reduced noise.

13. The scent presentation module according to claim 1, wherein
the airflow forming section causes air to flow into the scent holding section by linearly moving a pushing portion or a pump having a larger area of contact with the scent holding section than the pushing portion.

14. A scent presentation system, comprising
at least one scent presentation module that includes
a scent holding section that holds a scent,
an airflow forming section that forms a scent-containing airflow and that releases the airflow externally through a release opening of the scent presentation module,
a controller that controls an operation of the airflow forming section, and
a housing that includes therein the scent holding section, the airflow forming section, and the controller, the housing including, on an outer surface of the housing, a joining portion that enables the housing to be joined to another housing,
the scent presentation system presenting the scent using any of the at least one scent presentation module.

15. The scent presentation system according to claim 14, wherein
when a plurality of the modules is included, the modules are joined to each other.

16. The scent presentation system according to claim 14, wherein
the at least one scent presentation module is independently controllable.

17. The scent presentation system according to claim 14, wherein
the module further includes an air passage that circulates air from one of two ends of the module to another of the two ends, and
the scent presentation system supplies the air through the one of the two ends to generate an auxiliary airflow that assists the scent-containing airflow.

18. The scent presentation system according to claim 14, wherein
the scent presentation system is connected to at least one auxiliary airflow generating module, and generates an auxiliary airflow at any timing using the at least one auxiliary airflow generating module.

19. The scent presentation system according to claim 18, wherein
the auxiliary airflow generating module is connectable to the module.

20. The scent presentation system according to claim 14, wherein
the scent presentation system is connected to a deodorization unit, and performs deodorization operation at any timing using the deodorization unit.

21. The scent presentation system according to claim 14, wherein
the scent presentation system is connected to a charge unit, and charges the at least one scent presentation module at a time using the charge unit.

22. An information processing apparatus, comprising:
a terminal that is linked to a scent presentation module that includes a scent holding section that holds a scent, an airflow forming section that forms a scent-containing airflow and that releases the airflow externally through a release opening of the scent presentation module, a controller that controls an operation of the airflow forming section, and a housing that includes therein the scent holding section, the airflow forming section, and the controller, the housing including a joining portion that enables the housing to be joined to another housing; and
a processor
that receives information that is related to a state of the module and available to a user,
that performs processing of outputting and storing the information, and
that generates a signal related to the output and storing related to the information.

23. The information processing apparatus according to claim 22, wherein
the information processing apparatus
calculates an optimal condition for each scent presentation module on a basis of a piece of input information regarding the user and the piece of information related to the state of the module, the pieces of information being acquired using the terminal,
generates a drive signal on a basis of a result of the calculation, and
transmits the drive signal to the controller of the module.

24. A scent presentation module controlling system, comprising:
at least one scent presentation module that includes
a scent holding section that holds a scent,
an airflow forming section that forms a scent-containing airflow and that releases the airflow externally through a release opening of the scent presentation module,
a controller that controls an operation of the airflow forming section, and
a housing that includes therein the scent holding section, the airflow forming section, and the controller, the housing including a joining portion that enables the housing to be joined to another housing;
a terminal that is linked to the at least one scent presentation module; and
an information processing apparatus including a processor that receives information that is related to a state of the module and available to a user, that performs processing of outputting and storing the information, and that generates a signal related to the output and storing related to the information.
